(19) United States
(12) Patent Application Publication
(10) Pub. No.: US 2005/0107645 A1
(43) Pub. Date: May 19, 2005

(54) Fluorine-containing alcohol and method for its production

(75) Inventor: Yutaka Furukawa, Yokohama-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: ASAHI GLASS COMPANY LIMITED, Tokyo (JP)

(21) Appl. No.: 11/017,871

(22) Filed: Dec. 22, 2004

Related U.S. Application Data
(63) Continuation of application No. PCT/JP03/07771, filed on Jun. 19, 2003.

(30) Foreign Application Priority Data
Jun. 27, 2002 (JP) 2002-188062
Jul. 4, 2002 (JP) 2002-195905

(51) Int. Cl. C07C 31/34

(52) U.S. Cl. 568/842; 568/844

(57) ABSTRACT

A novel fluorine-containing alcohol and a method for its production.

A fluorine-containing alcohol represented by R¹CY¹HCY²Y³OQ²OH or HOQ²OCZ¹Z²CZ³HR²CZ⁴HCZ⁵Z⁶OQ²OH (wherein R¹ is a monovalent fluorine-containing organic group, etc., R² is a bivalent fluorine-containing organic group, etc., Y¹ to Y³ and Z¹ to Z⁶ are fluorine atoms, and Q¹ and Q² are alkylene groups, etc.). A method for producing a fluorine-containing alcohol having a group represented by —CX¹HCX²X³OQOH (wherein X¹, X² and X³ are fluorine atoms, and Q is an alkylene group, etc.), which comprises reacting a compound having a group represented by —CX¹═CX²X³ with a diol represented by HOQOH in the presence of an alkali metal compound.

### TECHNICAL FIELD

The present invention relates to a novel fluorine-containing alcohol and a method for its production.

### BACKGROUND ART

As a fluorine-containing compound having a terminal hydroxyl group, R^{f}(CF₂CF₂)ₙCH₂CH₂OH (R^{f} is a polyfluoroalkyl group) may be mentioned, and the fluorine-containing compound can be produced by the following method.

A method which comprises telomerizing R^{f}I and CF₂═CF₂ into R^{f}(CF₂CF₂)ₙI (n is an integer of at least 1), adding it to ethylene and converting the resulting R^{f}(CF₂CF₂)ₙCH₂CH₂I to R^{f}(CF₂CF₂)ₙCH₂CH₂OH by using a solid acid catalyst carrying an alkali metal (JP-A-2000-79345).

However, because R^{f}(CF₂CF₂)ₙI produced by telomerization reaction is a compound having a distribution in respect of the number of carbon atoms (i.e., n in the above chemical formula), R^{f}(CF₂CF₂)ₙCH₂CH₂OH derived from the compound is also a fluorine-containing compound having a distribution in respect of the number of carbon atoms. Accordingly, separation is required after the reaction in order to obtain a fluorine-containing compound having a terminal hydroxyl group and a specific number of carbon atoms.

The present inventors found that it is necessary to use a diol as a starting material for the reaction in order to solve the problem of the distribution in respect of the number of carbon atoms in the telomerization reaction, and found a novel compound having a terminal hydroxyl group based on the knowledge. Namely, the object of the present invention is to provide a novel fluorine-containing alcohol and a method for its production.

### DISCLOSURE OF THE INVENTION

The present invention provides a fluorine-containing alcohol represented by the following formula 1 (compound 1) or the following formula 2 (compound 2):
R¹CY¹HCY²Y³OQ¹OH Formula 1
HOQ²OCZ¹Z²CZ³HR²CZ⁴HCZ⁵Z⁶OQ³OH Formula 2
wherein the symbols in the formulae (1) and (2) have the following meanings:
R¹: a monovalent organic group, a halogen atom or a hydrogen atom;
R²: a bivalent organic group;
Y¹, Y² and Y³: independently hydrogen atoms or fluorine atoms provided that when R¹ is not a fluorine atom, at least one of Y¹, Y² and Y³ is a fluorine atom;
Z¹, Z², Z³, Z⁴, Z⁵ and Z⁶: independently hydrogen atoms or fluorine atoms provided that at least one of Z¹, Z² and Z³ is a fluorine atom, and at least one of Z⁴, Z⁵ and Z⁶ is a fluorine atom;
Q¹, Q² and Q³: independently bivalent organic groups.

Further, the present invention provides a method for producing a fluorine-containing alcohol (compound 5) having a group represented by the following formula (5), which comprises reacting a compound (compound 3) having a group represented by the following formula (3) and a diol (compound 4) represented by the following formula (4), in the presence of an alkali metal compound:
—CX¹═CX²X³ Formula 3
HOQOH Formula 4
—CX¹HCX²X³OQOH Formula 5
provided that the symbols in the formulae (3), (4) and (5) have the following meanings:
X¹, X² and X³: independently hydrogen atoms or fluorine atoms provided that at least one of X¹, X² and X³ is a fluorine atom;
Q: a bivalent organic group.

### BEST MODE FOR CARRYING OUT THE INVENTION

In compound 1, R¹ is a monovalent organic group, a halogen atom or a hydrogen atom. R¹ is preferably a monovalent hydrocarbon group or a monovalent halogenated hydrocarbon group, particularly preferably a monovalent halogenated hydrocarbon group containing an etheric oxygen atom. R¹ may be linear, branched or cyclic.

Especially, R¹ is preferably a fluorohydrocarbon group, particularly preferably a perfluorohydrocarbon group having all the hydrogen atoms of a hydrocarbon group substituted by fluorine atoms, and most preferably a perfluorohydrocarbon group containing an etheric oxygen atom which, if any, is preferably present at a terminal portion.

In the compound 1, Y¹, Y² and Y³ are preferably fluorine atoms, and it is preferred that all of them are fluorine atoms. Further, Q¹ is preferably a bivalent organic group, particularly preferably an alkylene group. Further, Q¹ is preferably a group represented by —(CH₂)ₜ— (t is an integer of at least 1), and t is preferably from 2 to 12, particularly preferably from 2 to 6.

The compound 1 is preferably a compound (compound 6) represented by R′CFHCF₂O(CH₂)ₘOH (wherein R′ is a monovalent fluorine-containing C₁₋₁₆ organic group containing an etheric oxygen atom, and m is an integer of from 1 to 12) because of ease of synthesis. In the compound 6, R′ may be linear or branched, and if branched, preferably has a branch preferably represented by (CF₃)₂CF— at an end.

Especially, R′ is preferably a fluorinated hydrocarbon group, particularly preferably a perfluorohydrocarbon group having all the hydrogen atoms of a hydrocarbon group substituted by fluorine atoms, most preferably a perfluorohydrocarbon group containing an etheric oxygen atom which, if any, is preferably present at a terminal portion.

Specific examples of the compound 1 are the following compounds.
H(CF₂)₂O(CH₂)₂OH,
H(CF₂)₂O(CH₂)₃OH,
H(CF₂)₂O(CH₂)₄OH,
H(CF₂)₂O(CH₂)₆OH,
CF₃CFHCF₂O(CH₂)₂OH,
CF₃CFHCF₂O(CH₂)₃OH,
CF₃CFHCF₂O(CH₂)₄OH,
CF₃CFHCF₂O(CH₂)₆OH,
C₂F₅CFHCF₂O(CH₂)₂OH.
C₂F₅CFHCF₂O(CH₂)₃OH,
C₂F₅CFHCF₂O(CH₂)₄OH,
C₂F₅CFHCF₂O(CH₂)₆OH,
C₃F₇CFHCF₂O(CH₂)₂OH,
C₃F₇CFHCF₂O(CH₂)₃OH,
C₃F₇CFHCF₂O(CH₂)₄OH,
C₃F₇CFHCF₂O(CH₂)₆OH,
C₅F₁₁CFHCF₂O(CH₂)₂OH,
C₅F₁₁CFHCF₂O(CH₂)₃OH,
C₅F₁₁CFHCF₂O(CH₂)₄OH,
C₅F₁₁CFHCF₂O(CH₂)₆OH,
C₇F₁₅CFHCF₂O(CH₂)₂OH,
C₇F₁₅CFHCF₂O(CH₂)₃OH,
C₇F₁₅CFHCF₂O(CH₂)₄OH,
C₇F₁₅CFHCF₂O(CH₂)₆OH.
C₃F₇OCFHCF₂O(CH₂)₂OH,
C₃F₇OCFHCF₂O(CH₂)₃OH,
C₃F₇OCFHCF₂O(CH₂)₄OH,
C₃F₇OCFHCF₂O(CH₂)₆OH,
C₄F₉OCFHCF₂OCH₂CH₂OH,
C₄F₉OCFHCF₂O(CH₂)₃OH,
C₄F₉OCFHCF₂O(CH₂)₄OH,
C₄F₉OCFHCF₂O(CH₂)₆OH,
C₅F₁₁OCFHCF₂O(CH₂)₂OH,
C₅F₁₁OCFHCF₂O(CH₂)₃OH,
C₅F₁₁OCFHCF₂O(CH₂)₄OH,
C₅F₁₁OCFHCF₂O(CH₂)₆OH,
C₆F₁₃OCFHCF₂O(CH₂)₂OH,
C₆F₁₃OCFHCF₂O(CH₂)₃OH,
C₆F₁₃OCFHCF₂O(CH₂)₄OH,
C₆F₁₃OCFHCF₂O(CH₂)₆OH.
(CF₃)₂CFCF₂CFHCF₂O(CH₂)₂OH,
(CF₃)₂CFCF₂CFHCF₂O(CH₂)₃OH,
(CF₃)₂CFCF₂CFHCF₂O(CH₂)₄OH,
(CF₃)₂CFCF₂CFHCF₂O(CH₂)₆OH,
F[CF(CF₃)CF₂O]CFHCF₂O(CH₂)₂OH,
F[CF(CF₃)CF₂O]CFHCF₂O(CH₂)₄OH,
F[CF(CF₃)CF₂O]₂CFHCF₂O(CH₂)₂OH,
F[CF(CF₃)CF₂O]₂CFHCF₂O(CH₂)₄OH,
F[CF(CF₃)CF₂O]₃CFHCF₂O(CH₂)₂OH, and
F[CF(CF₃)CF₂O]₃CFHCF₂O(CH₂)₄OH.

In the compound 2, R² is preferably a bivalent hydrocarbon group or a bivalent halogenated hydrocarbon group, particularly preferably a bivalent halogenated hydrocarbon group containing an etheric oxygen atom. R² may be linear, branched or cyclic.

Especially, R² is preferably a fluorinated hydrocarbon group, more preferably a perfluorohydrocarbon group having all the hydrogen atoms of a hydrocarbon group substituted by fluorine atoms, most preferably a perfluorohydrocarbon group containing an etheric oxygen atom which, if any, is preferably present at a terminal portion.

In the compound 2, Z¹, Z², Z³, Z⁴, Z⁵ and Z⁶ are preferably fluorine atoms, and it is preferred that all of them are fluorine atoms. Further, Q² and Q³ are preferably the same as mentioned for Q¹ in the compound 1.

The compound 2 is preferably a compound (compound 7) represented by R″ [CFHCF₂O(CH₂)_{q}OH]₂ (wherein R″ is a bivalent fluorine-containing C₁₋₁₆ organic group containing an etheric oxygen atom, and q is an integer of from 1 to 12) because of ease of synthesis. In the compound 7, R″ be linear or branched, and if branched, preferably has a branch preferably represented by (CF₃)₂CF— at an end.

Especially, R″ is preferably a fluorinated hydrocarbon group, more preferably a perfluorohydrocarbon group having all the hydrogen atoms of a hydrocarbon group substituted by fluorine atoms, most preferably a perfluorohydrocarbon group containing an etheric oxygen atom which, if any, is preferably present at a terminal portion.

Specific examples of the compound 2 are the following compounds.
HO(CH₂)₂OCF₂CFH(CF₂)₂CFHCF₂O(CH₂)₂OH,
HO(CH₂)₃OCF₂CFH(CF₂)₂CFHCF₂O(CH₂)₃OH,
HO(CH₂)₄OCF₂CFH(CF₂)₂CFHCF₂O(CH₂)₄OH,
HO(CH₂)₆OCF₂CFH(CF₂)₂CFHCF₂O(CH₂)₆OH,
HOCH₂CH₂OCF₂CFH(CF₂)₃CFHCF₂OCH₂CH₂OH,
HO(CH₂C)₃OCF₂CFH(CF₂)₃CFHCF₂O(CH₂)₃OH,
HO(CH₂)₄OCF₂CFH(CF₂)₃CFHCF₂O(CH₂)₄OH,
HO(CH₂)₆OCF₂CFH(CF₂)₃CFHCF₂O(CH₂)₆OH.
HO(CH₂)₂OCF₂CFHO(CF₂)₂OCFHCF₂O(CH₂)₂OH,
HO(CH₂)₃OCF₂CFHO(CF₂)₂OCFHCF₂O(CH₂)₃OH,
HO(CH₂)₄OCF₂CFHO(CF₂)₂OCFHCF₂O(CH₂)₄OH,
HO(CH₂)₆OCF₂CFHO(CF₂)₂OCFHCF₂O(CH₂)₆OH,
HO(CH₂)₂OCF₂CFHO(CF₂)₃OCFHCF₂O(CH₂)₂OH,
HO(CH₂)₃OCF₂CFHO(CF₂)₃OCFHCF₂O(CH₂)₃OH,
HO(CH₂)₄OCF₂CFHO(CF₂)₃OCFHCF₂O(CH₂)₄OH,
HO(CH₂)₆OCF₂CFHO(CF₂)₃OCFHCF₂O(CH₂)₆OH,
HO(CH₂)₂OCF₂CFH[OCF₂CF(CF₃)]OCF₂(CF₂)₄CF₂O[CF(CF₃)CF₂O]C FHCF₂O(CH₂)₂OH.
HO(CH₂)₃OCF₂CFH[OCF₂CF(CF₃)]OCF₂(CF₂)₄CF₂O[CF(CF₃)CF₂O]C FHCF₂O(CH₂)₃OH,
HO(CH₂)₄OCF₂CFH[OCF₂CF(CF₃)]OCF₂(CF₂)₄CF₂O[CF(CF₃)CF₂O]C FHCF₂O(CH₂)₄OH and
HO(CH₂)₆OCF₂CFH[OCF₂CF(CF₃)]OCF₂(CF₂)₄CF₂O[CF(CF₃)CF₂O]C FHCF₂O(CH₂)₆OH.

In the compound 3, X¹, X² and X³ are preferably fluorine atoms, and it is preferred that all of them are fluorine atoms. The compound 3 can be prepared by the method disclosed in J. Am. Chem. Soc, 75, 4525 (1953) or the like.

The compound 3 is preferably a compound (compound 8) represented by the following formula 8.
R[CX¹═CX²X³]ₚ Formula 8
R: a p-valent organic group;
p: an integer of from 1 to 4;
X¹, X² and X³: independently hydrogen atoms or fluorine atoms provided that at least one of X¹, X² and X³ is a fluorine atom.

In the compound 8, R is preferably the same as mentioned for R¹ in the compound 1 or for R² in the compound 2. X¹, X² and X³ are preferably a fluorine atom, and it is preferred that all of them are fluorine atoms. p is preferably 1 or 2.

Specific examples of the compound 3 are the following compounds.
CF₂═CF₂, CF₃CF═CF₂, C₂F₅CF═CF₂, C₃F₇CF═CF₂, C₅F₁₁CF═CF₂, C₇F₁₅CF═CF₂, C₃F₇OCF═CF₂, C₄F₉OCF═CF₂, C₅F₁₁OCF═CF₂, C₆F₁₃OCF═CF₂, (CF₃)₂CFCF₂CF═CF₂, F[CF(CF₃)CF₂O]CF═CF₂, F[CF(CF₃)CF₂O]₂CF═CF₂, F[CF(CF₃)CF₂O]₃CF═CF₂. CF₂═CF(CF₂)₂CF═CF₂, CF₂═CF(CF₂)₃CF═CF₂, CF₂═CFO(CF₂)₂OCF═CF₂, CF₂═CFO(CF₂)₃OCF═CF₂ and CF₂═CF[OCF₂CF(CF₃)]OCF₂ (CF₂)₄CF₂O[CF(CF₃)CF₂O]CF═CF₂.

In the compound 4, Q is preferably the same as mentioned for Q¹ in the compound 1.

As specific examples of the compound 4, HO(CH₂)₂OH, HO(CH₂)₃OH, HO(CH₂)₄OH and HO(CH₂)₆OH are preferably mentioned.

In the compound 5, X¹, X², X³ and Q are preferably the same as those in the compound 3 or 4.

The compound 5 is preferably a compound (compound 9) represented by the following formula 9.
R[CX¹HCX²X³ OQOH]ₚ formula 9
R: a p-valent organic group;
X¹, X² and X³: independently hydrogen atoms or fluorine atoms provided that at least one of X¹, X² and X³ is a fluorine atom;
Q: a bivalent organic group;
P: an integer of from 1 to 4.

In the compound 9, R, X¹, X², X³, Q and p are preferably the same as those in the compound 4 or the compound 8. The compound 5 is preferably the compound 1 or the compound 2.

The alkali metal compound to be used in the reaction of the compound 3 and the compound 4 is preferably an alkali metal, an alkali metal hydride, an alkali metal hydroxide, an alkali metal amide or the like. Specifically, an alkali metal such as Na, K or Cs, an alkali metal hydroxide such as NaOH or KOH, an alkali metal hydride such as NaH or KH, or an alkali metal amide such as NaNH₂, KNH₂ is preferably mentioned.

In the reaction, the alkali metal compound is preferably used in an amount of from 0.01 to 1.0 mol, particularly preferably in an amount of from 0.05 to 0.5 mol in view of the reaction rate, based on 1 mol of the compound 3, though there is no particular restriction. The usage range is preferred because the reaction proceeds at an appropriate reaction rate with little production of by-products.

In the reaction, the compound 4 is preferably used in an amount of from 1 to 5 mols, particularly preferably from 2 to 4 mols based on 1 mol of the compound 3 for production of the compound having a group represented by the formula 5, namely the compound 1, and is preferably used in an amount of from 2 to 10 mols, particularly preferably from 4 to 8 mols based on 1 mol of the compound 3 for production of the compound having two groups represented by the formula 5, namely the compound 2. The reaction is preferably carried out in the range because the compound 3 is likely to react with only one hydroxyl group in the compound 4 to give the product.

Further, the temperature for the reaction is preferably from 0 to 150° C., particularly preferably from 40 to 120° C. The reaction is preferably carried out within this temperature range because the reaction proceeds at an appropriate reaction rate without homopolymerization of the compound 3.

Further, in the reaction, a solvent may or may not be used, but it is preferably used. The solvent is preferably a solvent which dissolves the compound 4, a solvent which dissolves the compound 5, or a solvent which is practically inert in the reaction. The solvent is preferably an ether, a nitrile compound or the like, and specifically, it is preferably diethyl ether, glyme, dioxane, tetrahydrofuran, acetonitrile or propionitrile, particularly preferably dioxane, tetrahydrofuran or acetonitrile.

The solvent is preferably used in such an amount that the compound 5 as the product accounts for from 1 to 60 mass %, particularly from 3 to 50 mass % in view of the reaction rate and the productivity, though there is no particular restriction.

Further, the compound 3 tends to polymerize at high pressure, and therefore in order to prevent the polymerization, a polymerization inhibitor is preferably used in the reaction. The polymerization inhibitor may be put in the reaction system before or with the starting materials. The polymerization inhibitor is preferably limonene, pinene, cymene, terpinene or the like, though there is no particular restriction.

In the present invention, the compound 5 is presumed to be produced by the following mechanism.

Namely, the alkali metal compound converts the diol (compound 4) to an alkoxide compound, then the alkoxide compound is added to the compound 3, and the metal portion is replaced by a hydrogen atom to produce the compound 5. The use of a diol allows production of a fluorine-containing alcohol because one hydroxyl group remains after the other hydroxyl group is reacted.

According to the present invention, a novel fluorine-containing alcohol can be produced. The fluorine-containing alcohol is useful as an intermediate of cleaning agents or various compounds. For example, a fluorine-containing acrylate obtained by reacting the compound 1 with acrylic acid is useful as a starting-material for a UV curable resin or a water and oil repellent. Further, when used as a comonomer component for a condensation resin such as a urethane, the compound 2 can alter the surface properties of the resin.

Further, the compound 3 as the starting material for the fluorine-containing alcohol of the present invention can be prepared by the direct fluorination disclosed in Adv. Synth. Catal. 2001, 343, No. 2, which allows a choice of the structure of the starting material and therefore can give fluorine-containing alcohols in various structures.

### EXAMPLES

Now, the present invention will be described in detail with reference to Examples, but it should be understood that the present invention is by no means restricted thereto.

### Example 1

30 g of 1,4-dioxane, 10 g of C₃F₇OCF═CF₂, 4.67 g of HO(CH₂)₂OH and 0.55 g of KOH were sealed in a stainless steel reaction vessel having a capacity of 50 mL, then reacted at 70° C. for 8 hours with stirring, and after addition of 50 mL of water, allowed to separate into two layers. Then, the organic layer was distilled to obtain 10.9 g of CF₃CF₂CF₂OCFHCF₂OCH₂CH₂OH and C₃F₇OCFHCF₂OCH₂CH₂OCF₂CFHOC₃F₇ as the products in the ratio of 75:25 (molar ratio).

The results of analysis of CF₃CF₂CF₂OCFHCF₂OCH₂CH₂OH are shown below.

IR (neat): 3395, 1342, 1236, 1199, 1153, 1102, 985 cm⁻¹,

¹H-NMR(CDCl₃)δ: 1.84 (t, J=6.3 Hz, 3H, OH), 3.86-3.94 (m, 2H, CH₂OH), 4.15 (t, J=4.6 Hz, 2H, CF₂OCH₂), 5.95 (d, t, 53.5 Hz, 2.9 Hz, 1H, CFHCF₂),

¹⁹F-NMR(CDCl₃)δ: −81.4 (t, J=6.4 Hz, 3F, CF₃), −84.88 and −86.99 (ABquartet, J=145.1 Hz, 2F, CF₂OCFH), −89.00 and −89.84 (ABquartet, J=144.0 Hz, 2F, CF₂OCH₂), −129.50-−129.60 (m, 2F, CF₂CF₃), −144.0 (d, quintet, J=53.5 Hz, 6.4 Hz, 1F, CFH).

### Example 2

30 g of 1,4-dioxane, 10 g of C₃F₇OCF═CF₂, 13.55 g of HO(CH₂)₄OH and 0.55 g of KOH were sealed in a stainless steel reaction vessel having a capacity of 50 mL, reacted at 70° C. for 8 hours with stirring, and after addition of 50 mL of water, allowed to separate into two layers. Then, the organic layer was distilled to obtain 12.8 g of CF₃CF₂CF₂OCFHCF₂O(CH₂)₄OH and C₃F₇OCFHCF₂O(CH₂)₄OCF₂CFHOC₃F₇ as the products in the ratio of 94:6 (molar ratio).

The results of analysis of CF₃CF₂CF₂OCFHCF₂O(CH₂)₄OH are shown below.

IR (neat): 3349, 2951, 1341, 1237, 1199, 1153, 987 cm⁻¹,

¹H-NMR(CDCl₃)δ: 1.39 (s, 1H, OH), 1.60-1.73 (m, 2H, CH₂CH₂OH), 1.73-1.86 (m, 2H, CF₂OCH₂CH₂), 3.63-3.76 (m, 2H, CH₂OH), 4.03 (t, J=6.3 Hz, 2H, CF₂OCH₂), 5.86 (d, t, 53.7 Hz, 2.8 Hz, 1H, CFHCF₂),

¹⁹F-NMR(CDCl₃)δ: −81.4 (t, J=7.5 Hz, 3F, CF₃), −84.86 and −86.96 (ABquartet, J=145.0 Hz, 2F, CF₂OCFH), −89.30 and −89.94 (ABquartet, J=144.0 Hz, 2F, CF₂OCH₂), −129.5-−129.6 (m, 2F, CF₂CF₃), −144.2 (d, quintet, J=53.7 Hz, 8.6 Hz, 1F, CFH).

### Example 3

30 g of 1,4-dioxane, 10 g of C₃F₇OCF═CF₂, 5.72 g of HOCH₂CH₂CH₂OH and 0.55 g of KOH were sealed in a stainless steel reaction vessel having a capacity of 50 mL, reacted at 70° C. for 8 hours with stirring, and after addition of 50 mL of water, allowed to separate into two layers. Then, the organic layer was distilled to obtain 11.6 g of CF₃CF₂CF₂OCFHCF₂OCH₂CH₂CH₂OH and C₃F₇OCFHCF₂OCH₂CH₂CH₂OCF₂CFHOC₃F₇ as the products in the ratio of 89:11 (molar ratio).

The results of analysis of CF₃CF₂CF₂OCFHCF₂OCH₂CH₂CH₂OH are shown below.

IR(neat): 3356, 1342, 1236, 1199, 1153, 1098, 987 cm⁻¹,

¹¹H-NMR(CDCl₃)δ: 1.58 (s, 1H, OH), 1.93 (quintet, J=6.1 Hz, 2H, CH₂CH₂CH₂), 3.76 (t, J=6.0 Hz, 2H, CH₂OH), 4.14 (t, J=6.1 Hz, 2H, CF₂OCH₂), 5.87 (d, t, 53.5 Hz, 2.9 Hz, 1H, CFHCF₂),

¹⁹F-NMR (CDCl₃)δ: −81.4 (t, J=7.5 Hz, 3F, CF₃), −84.88 and −87.02 (ABquartet, J=148.0 Hz, 2F, CF₂OCFH), −89.36 and −90.05 (ABquartet, J=146.1 Hz, 2F, CF₂OCH₂), −129.54-−129.64 (m, 2F, CF₂CF₃), −144.2 (d, quintet, J=53.5 Hz, 8.1 Hz, 1F, CFH).

### Example 4

30 g of 1,4-dioxane, 10 g of C₃F₇OCF═CF₂, 8.88 g of HO(CH₂)₆OH and 0.55 g of KOH were sealed in a stainless steel reaction vessel having a capacity of 50 mL, reacted at 70° C. for 8 hours with stirring, and after addition of 50 mL of water, allowed to separate into two layers. Then, the organic layer was distilled to obtain 13.0 g of CF₃CF₂CF₂OCFHCF₂O(CH₂)₆OH and C₃F₇OCFHCF₂O(CH₂)₆OCF₂CFHOC₃F₇ as the products in the ratio of 87:13 (molar ratio).

The results of analysis of CF₃CF₂CF₂OCFHCF₂O(CH₂)₆OH are shown below.

IR(neat): 3351, 2942, 1341, 1237, 1199, 1154, 1092, 988 cm⁻¹,

¹H-NMR(CDCl₃)δ: 1.32-1.48 (5H, OH, CH₂CH₂CH₂CH₂CH₂CH₂), 1.52-1.75 (m, 4H, CH₂CH₂CH₂CH₂CH₂CH₂), 3.65 (t, J=6.5 Hz, 2H, CH₂OH), 3.98 (t, J=6.5 Hz, 2H, CF₂OCH₂), 5.85 (d, t, 54.1 Hz, 2.9 Hz, 1H, CFHCF₂),

¹⁹F-NMR (CDCl₃)δ: −81.3 (t, J=6.4 Hz, 3F, CF₃), −84.71 and −86.86 (ABquartet, J=146.1 Hz, 2F, CF₂OCFH), −89.06 and −89.85 (ABquartet, J=144.0 Hz, 2F, CF₂OCH₂), −129.38 −129.50 (m, 2F, CF₂CF₃), −144.1 (d, t, J=54.1 Hz, 1F, CFH).

### Example 5

30 g of 1,4-dioxane, 10 g of CF₂═CFO(CF₂)₂OCF═CF₂, 12.26 g of HO(CH₂)₄OH and 0.55 g of KOH were sealed in a stainless steel reaction vessel having a capacity of 50 mL, reacted at 70° C. for 8 hours with stirring, and after addition of 50 mL of water, allowed to separate into two layers. Then, the organic layer was distilled to obtain 13.2 g of HO(CH₂)₄OCF₂HCFO(CF₂)₂OCFHCF₂O(CH₂)₄OH as the product.

The results of analysis of HO(CH₂)₄OCF₂HCFO(CF₂)₂OCFHCF₂O(CH₂)₄OH are shown below.

IR(neat): 3349, 2951, 1341, 1237, 1199, 1153, 987 cm⁻¹,

¹H-NMR(CDCl₃)δ: 1.39 (s, 2H, OH), 1.60-1.73 (m, 4H, CH₂CH₂OH), 1.73-1.86 (m, 4H, CF₂OCH₂CH₂), 3.63-3.76 (m, 4H, CH₂OH), 4.03 (t, J=6.3 Hz, 4H, CF₂OCH₂), 5.86 (d, t, 53.7 Hz, 2.8 Hz, 2H, CFHCF₂),

¹⁹F-NMR (CDCl₃)δ: −84.86 and −86.96 (ABquartet, J=145.0 Hz, 4F, CF₂OCFH), −89.30 and −89.94 (ABquartet, J=144.0 Hz, 4F, CF₂OCH₂), −144.2 (d, quintet, J=53.7 Hz, 8.6 Hz, 2F, CFH).

The entire disclosures of Japanese Patent Application No. 2002-188062 filed on Jun. 27, 2002 and Japanese Patent Application No. 2002-195905 filed on Jul. 4, 2002 including specifications, claims and summaries are incorporated herein by reference in their entireties.

1. A fluorine-containing alcohol represented by the following formula (1) or (2):
R¹CY¹HCY²Y³OQ¹OH Formula 1
HOQ²OCZ¹Z²CZ³HR²CZ⁴HCZ⁵Z⁶OQ³OH Formula 2
wherein the symbols in the formulae (1) and (2) have the following meanings:
R¹: a monovalent organic group, a halogen atom or a hydrogen atom;
R²: a bivalent organic group;
Y¹, Y² and Y³: independently hydrogen atoms or fluorine atoms provided that when R¹ is not a fluorine atom, at least one of Y¹, Y² and Y³ is a fluorine atom;
Z¹, Z², Z³, Z⁴, Z⁵ and Z⁶: independently hydrogen atoms or fluorine atoms provided that at least one of Z¹, Z² and Z³ is a fluorine atom, and at least one of Z⁴, Z⁵ and Z⁶ is a fluorine atom;
Q¹, Q² and Q³: independently bivalent organic groups.

2. A method for producing a fluorine-containing alcohol having a group represented by the following formula (5), which comprises reacting a compound having a group represented by the following formula (3) and a diol represented by the following formula (4), in the presence of an alkali metal compound:
—CX¹═CX²X³ Formula 3
HOQOH Formula 4
—CX¹HCX²X³OQOH Formula 5
wherein the symbols in the formulae (3), (4) and (5) have the following meanings:
X¹, X² and X³: independently hydrogen atoms or fluorine atoms provided that at least one of X¹, X² and X³ is a fluorine atom;
Q: a bivalent organic group.

3. The fluorine-containing alcohol according to claim 1, wherein R¹ in the formula (1) is a monovalent perfluorohydrocarbon group containing an etheric oxygen atom, and R² in the formula (2) is a bivalent perfluorohydrocarbon group containing an etheric oxygen atom.

4. The fluorine-containing alcohol according to claim 1, wherein Q¹ in the formula (1), and Q² and Q³ in the formula (2) are —(CH₂)ₜ— (t is an integer of at least 1).

5. The fluorine-containing alcohol according to claim 1, wherein Y¹, Y², Y³, Z¹, Z², Z³, Z⁴, Z⁵ and Z⁶ in the formulae (1) and (2) are fluorine atoms.

6. The method for producing a fluorine-containing alcohol according to claim 3, wherein Q in the formula (5) is —(CH₂)ₜ— (t is an integer of at least 1).

7. The method for producing a fluorine-containing alcohol according to claim 3, wherein X¹, X² and X³ in the formula (5) are fluorine atoms.
